(19) United States
(12) Patent Application Publication
(10) Pub. No.: US 2009/0156843 A1
(43) Pub. Date: Jun. 18, 2009

(54) PROCESS FOR AROMATIZING 19-NORANDROST-4-EN-3-ONES TO ESTRA-1,3,5(10)-TRIENES

(76) Inventors: Michael SANDER, Frechen (DE); Jorg Gries, Haan (DE); Armin Schutz, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/334,048

(22) Filed: Dec. 12, 2008

Related U.S. Application Data
(60) Provisional application No. 61/028,679, filed on Feb. 14, 2008.

(30) Foreign Application Priority Data
Dec. 13, 2007 (EP) 0707613.6

(51) Int. Cl. C07J 75/00

(52) U.S. Cl. 552/625

(57) ABSTRACT

The present invention relates to a process for aromatizing 19-norandrost-4-en-3-ones (formula (II)) to astra-1,3,5(10)-trienes (formula (I))

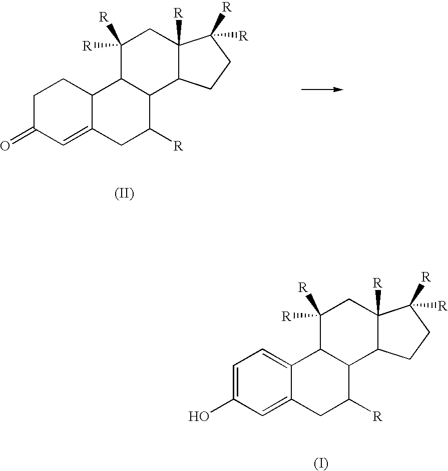

This application claims the benefit of the filing date of U.S. Provisional Application Ser. No. 61/028,679 filed Feb. 14, 2008.

The present invention relates to a process for aromatizing 19-nor-androst-4-en-3-ones (formula (II)) to estra-1,3,5(10)-trienes (formula (I)) according to scheme 1

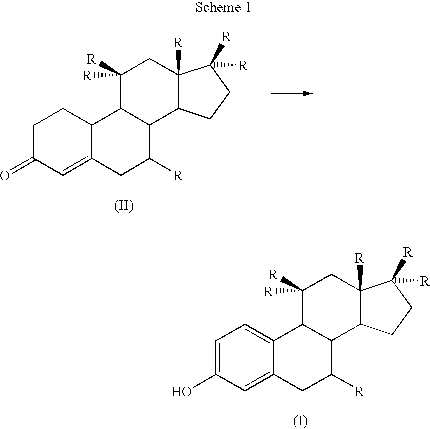

where, in formula (II) and (I), each R is independently any chemically stable radical.

The invention is based on the following definitions:

Cₙ-Alkyl:

Monovalent, straight-chain or branched, saturated hydrocarbon radical having n carbon atoms.

Cₙ-Alkylene:

Divalent, straight-chain or branched, saturated hydrocarbon radical having n carbon atoms.

Cₙ-Alkenyl:

Monovalent, straight-chain or branched hydrocarbon radical having n carbon atoms and at least one double bond.

Cₙ-Alkynyl:

Monovalent, straight-chain or branched hydrocarbon radical having n carbon atoms and at least one triple bond.

Cₙ-Cycloalkene

Cyclic monounsaturated hydrocarbon ring having n carbon atoms.

Cₙ-Alkoxy:

Straight-chain or branched Cₙ-alkyl ether radical of the formula —OR where R═Cₙ-alkyl.

Cₙ-Alkenoxy:

Straight-chain or branched Cₙ-alkenyl ether radical of the formula —OR where R═Cₙ-alkenyl.

Cₙ-Acyloxy:

Cₙ-Acyloxy is a linear or branched Cₙ-alkyl ester radical of the formula —O—C(O)—Cₙ-alkyl.

In general, n is 1 to 6, preferably 1 to 4 and more preferably 1 to 3.

Preferred examples include:

Acetyloxy and propanoyloxy.

Cₙ-Alkyloxycarbonyl

Cₙ-Alkyloxycarbonyl is the —C(O)—O—Cₙ-alkyl group.

In general, n is 1 to 6, preferably 1 to 5 and more preferably 1 to 4.

Cₙ-Aromatic

Cₙ-Aromatic is an aromatic ring system without a heteroatom and with n carbon atoms.

C₆-Aromatic is benzene; C₁₀-aromatic is naphthalene.

Cₙ-Aryl

Cₙ-Aryl is a monovalent aromatic ring system without a heteroatom and with n carbon atoms.

C₆-Aryl is phenyl. C₁₀-Aryl is naphthyl.

Cₙ-Aryloxy

Cₙ-Aryloxy is a Cₙ-aryl ether of the formula —O—Cₙ-aryl.

Preference is given to phenyloxy.

Heteroatoms

Heteroatoms are understood to mean oxygen, nitrogen or sulphur atoms.

Heteroaromatic

Heteroaromatic is an aromatic ring system having at least one heteroatom other than carbon. The heteroatoms which may occur are nitrogen atoms, oxygen atoms and/or sulphur atoms.

A monocyclic heteroaromatic according to the present invention has 5 or 6 ring atoms.

Heteroaromatics having 5 ring atoms include, for example, the rings: thiophene, thiazole, furan, pyrrole, oxazole, imidazole, pyrazole, isoxazole, isothiazole, oxadiazole, triazole, tetrazole and thiadiazole.

Heteroaromatics with 6 ring atoms include, for example, the rings: pyridine, pyridazine, pyrimidine, pyrazine and triazine.

Heterocycle

Heterocycle in the context of the invention is a fully hydrogenated heteroaromatic (fully hydrogenated heteroaromatic=saturated heterocycle), i.e. a nonaromatic ring system having at least one heteroatom other than a carbon. The heteroatoms which may occur are nitrogen atoms, oxygen atoms and/or sulphur atoms.

Heterocycles having 5 ring atoms include, for example, the rings: pyrrolidine, imidazolidine, pyrazolidine and tetrahydrofuran. Heterocycles having 6 ring atoms include, for example, the rings: piperidine, piperazine, morpholine, tetrahydropyran and thiomorpholine.

Unsaturated Heterocycle

Heterocycle having at least one double bond, where the ring system is not aromatic.

Heterocyclyl

Heterocycle with a free bonding valence.

Halogen

The term halogen encompasses fluorine, chlorine, bromine and iodine.

Preference is given to bromine.

Protecting Group

A group known to those skilled in the art, especially from Protective Groups in Organic Chemistry, Third Edition, Theodora W. Greene and Peter G. M. Wuts, which protects a functional group in subsequent reaction steps.

For example, Cₙ-alkyl, Cₙ-alkenyl or Cₙ-alkynyl radical, Cₙ-alkylcarbonyl, Cₙ-alkyloxycarbonyl or trialkylsilyl radicals or heterocyclyl rings are capable of protecting an oxygen function.

Trialkylsilyl

A trialkylsilyl radical represents the —SiR¹, R²R³ group where R¹, R², R³ are 3 identical or else different Cₙ-alkyl radicals. In general, n is 1 to 6, preferably 1 to 4; particularly preferred examples include: trimethylsilyl and tert-butyldimethylsilyl.

The invention relates more particularly to a process for preparing estra-1,3,5(10)-trienes of the formula (Ia) from compounds of the formula (IIa) according to scheme 2

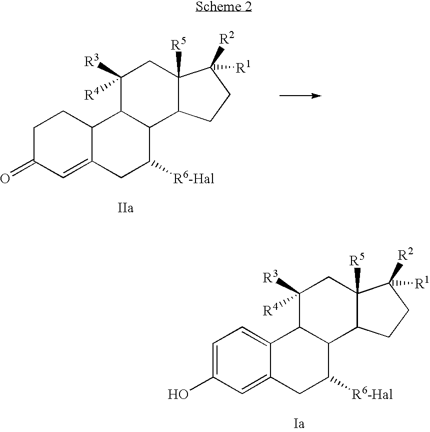

where, in the formulae (IIa) and (Ia),

R¹ and R² together form a keto group,

or
R¹ is hydrogen, a C₁-C₆-alkyl, C₂-C₆-alkenyl or C₂-C₆-alkynyl radical, or is an at least partly fluorinated C₁-C₆-alkyl radical, and
R² is hydrogen, hydroxyl, C₁-C₆-alkoxy radical or a C₁-C₆-acyloxy radical;
R³ and R⁴ are each independently hydrogen, halogen and/or —OR^{B} where R^{B} is hydrogen or a protecting group,
R⁵ is a methyl or ethyl radical, and
R⁶-Hal is a C₃-C₁₃-alkyl, C₃-C₁₃-alkenyl or C₃-C₁₃-alkynyl radical, in each case at least monosubstituted by halogen,
   and enantiomers and diastereomers thereof.

The process according to the invention is particularly suitable for preparing compounds of the general formula (Ib) from compounds of the general formula (IIb) according to scheme 3,

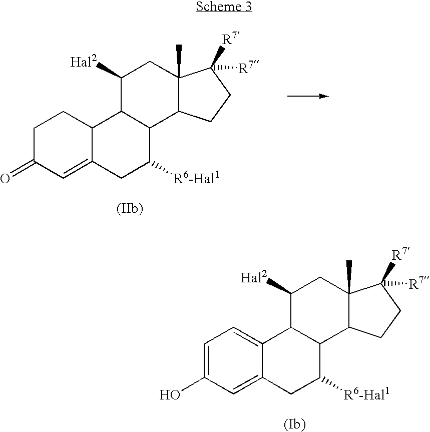

where, in the formulae (IIb) and (Ib),
Hal² is F or Cl and is bonded to the estratriene base skeleton in the 11β position,
R^{7′} and R^{7″} together form a keto group,
   or
R^{7′} is the —OR^{C} group where R^{C} is hydrogen or a protecting group,
R^{7″} is a C₁-C₄-alkyl radical or is an at least partly fluorinated C₁-C₄-alkyl radical,
   where R^{7′} is bonded to the estratriene base skeleton in the 17β position and R^{7″} in the 17α position, and
R⁶-Hal¹ is bonded to the estratriene base skeleton in the 7α position and is a C₃-C₁₁-alkyl radical which is at least monosubstituted by Hal¹ where Hal¹ is a chlorine, bromine or iodine atom,
   and enantiomers and diastereomers thereof.

Compounds of the formula (IIb) or (Ib) in which R^{7′} and R^{7″} together form a keto group are referred to hereinafter as (IIb)-17-Keto compounds and (Ib)-17-Keto compounds respectively, and are summarized in the formula (IIb-17-Keto) and (Ib-17-Keto) respectively.

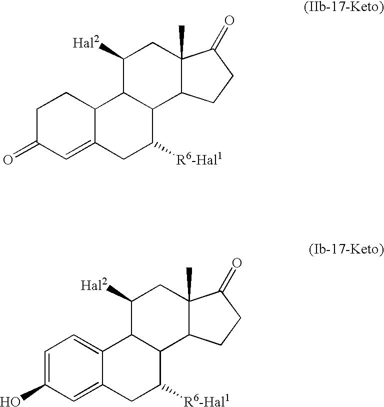

Compounds of the formula (IIb) and (Ib) in which R^{7′} is the —OR^{C} group where Rc is hydrogen or a protecting group, and R^{7″} is a C₁-C₄-alkyl radical or an at least partly fluorinated C₁-C₄-alkyl radical, are referred to hereinafter as (IIb-17β-OR^{C}) compounds and (Ib-17β-OR^{C}) compounds respectively, and are summarized in the formula (IIb-17-β-OR^{C}) and (Ib-17β-OR^{C}) respectively.

In IIb-17β-OH compounds and Ib-17β-OH compounds, the hydroxyl group in position 17 is present in free form.

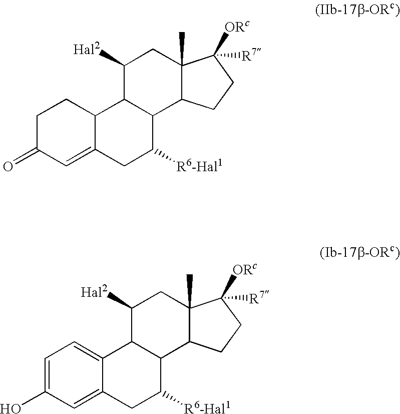

In the formulae (IIb-17-Keto), (Ib-17-Keto), (IIb-17β-OR^{C}) and (Ib-17β-OR^{C}), R⁶-Hal¹ and Hal² are each as defined in scheme 3.

The process according to the invention is very particularly suitable for preparing compounds of the general formula (Ic) from compounds of the general formula (IIc) according to scheme 4

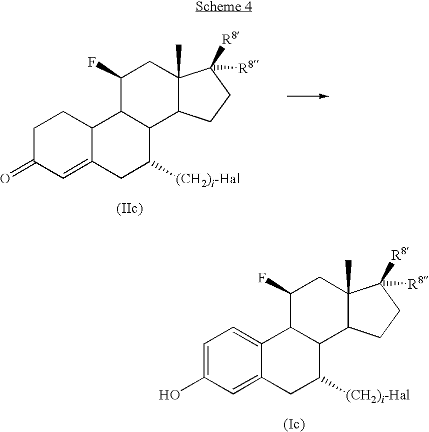

where, in the formulae (IIc) and (Ic),

R^{8′} and R^{8″} together form a keto group

or

R^{8′} is the —OR^{D} group where R^{D} is hydrogen or a protecting group,

R^{8″} is a methyl group,

i is an integer from 3 to 13 and

Hal is a chlorine, bromine or iodine atom,

and enantiomers and diastereomers thereof.

Compounds of the formula (IIc) or (Ic) in which R^{8′} and R^{8″} together form a keto group are referred to hereinafter as (IIc)-17-Keto compounds and (Ic)-17-Keto compounds respectively, and are summarized in the formula (IIc-17-Keto) and (Ic-17-Keto) respectively.

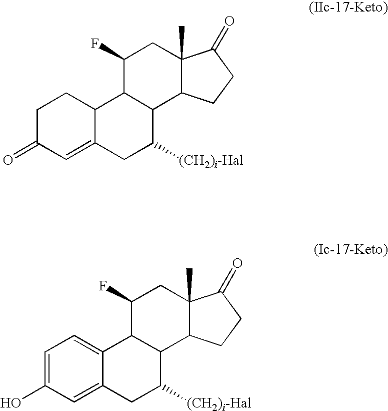

Compounds of the formula (IIc) or (Ic) in which R⁸ is the —OR^{D} group where R^{D} is hydrogen or a protecting group, and R⁸ is a methyl group, are referred to hereinafter as (IIc-17β-OR^{D}) compounds and (Ic-17β-OR^{D}) compounds respectively, and are summarized in the formula (IIc-17β-OR^{D}) and (Ic-17β-OR^{D}) respectively.

In IIc-17β-OH compounds and Ic-17β-OH compounds, the hydroxyl group in position 17 is present in free form.

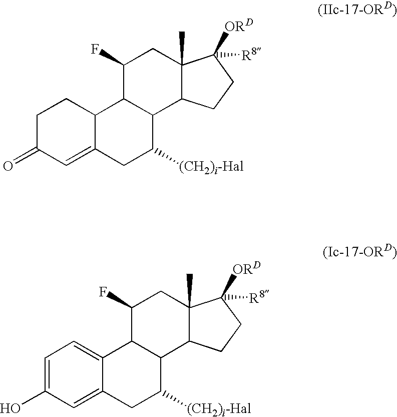

In the formulae (IIb-17-Keto), (Ib-17-Keto), (IIb-17βOR^{D}) and (Ib-17β-OR^{D}), R^{8″}, Hal, R^{D} and i are each as defined in scheme 4.

From compounds of the general formula I, it is possible to prepare compounds with high antiestrogenic activity.

For instance, WO 03/045972 describes estrogen antagonists which display their antiestrogenic activity owing to the competitive displacement of the natural estrogens from their receptor and/or through destabilization of the estrogen receptor. In the latter case, reference is also made to selective estrogen receptor destabilizers (SERDs). In both cases, the transmission of the estrogenic stimulus is suppressed. The degradation of the estrogen receptor can also contribute to the antiestrogenic action (Selective Estrogen Receptor Degradation).

The antiestrogenic compounds preferably have only a low residual estrogenic action, if any.

The inventive aromatization is suitable, inter alia, for the preparation of compounds from WO 03/045972.

WO 03/045972 discloses, inter alia, compounds of the formula (Vb). These can be prepared according to scheme 5

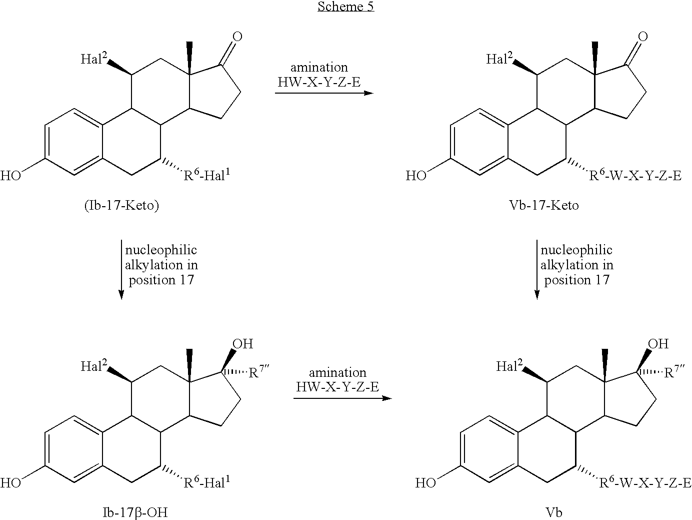

where R^{7″}, Hal² and R⁶-Hal¹ are each as defined in scheme 3 and
W is —N(R⁷)— where R⁷ is hydrogen or a C₁-C₄-alkyl radical,
X is —(CH₂)_{q}— where q=0 or an integer of 1-12,
Y is a direct bond between X and Z or is a —SOₙ— group where n=0, 1 or 2,
Z is a straight- or branched-chain C₁-C₇-alkylene radical which is at least partly fluorinated,
E is —CF₃ or is pentafluorophenyl.

Accordingly, compounds of the formula (Ib-17-Keto) can first be aminated with amines of the formula H—W—X—Y-Z-E to obtain compounds of the formula (Vb-Keto). Subsequently, the compounds of the formula (Vb-Keto) are converted in a nucleophilic alkylation reaction in position 17 to obtain compounds of the formula (Vb).

Alternatively and preferably, compounds of the formula (Ib-17-Keto) are first converted by a nucleophilic alkylation reaction in position 17 to compounds of the formula (Ib-17β-OH). Subsequently, the compounds of the formula (Ib-17β-OH) are aminated with amines of the formula H—W—X—Y-Z-E to obtain compounds of the formula (Vb).

The alkyl group in the 17α position can in each case be introduced by customary alkylating reagents, for example Grignard reagents (Cₙ-alkyl-Mg-Hal) or alkyllithium compounds (Cₙ-alkyl-Li).

The process according to the invention is particularly advantageous for the preparation of the compound AE1:

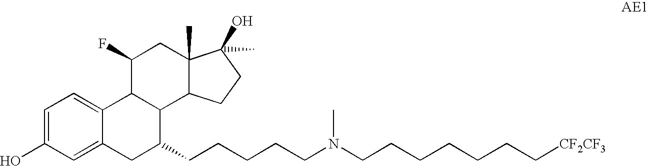

This compound is likewise described for the first time in WO 03/045972.

The preferred compounds of the AE1 type (compounds of the formula (Vc)) can be prepared according to scheme 6

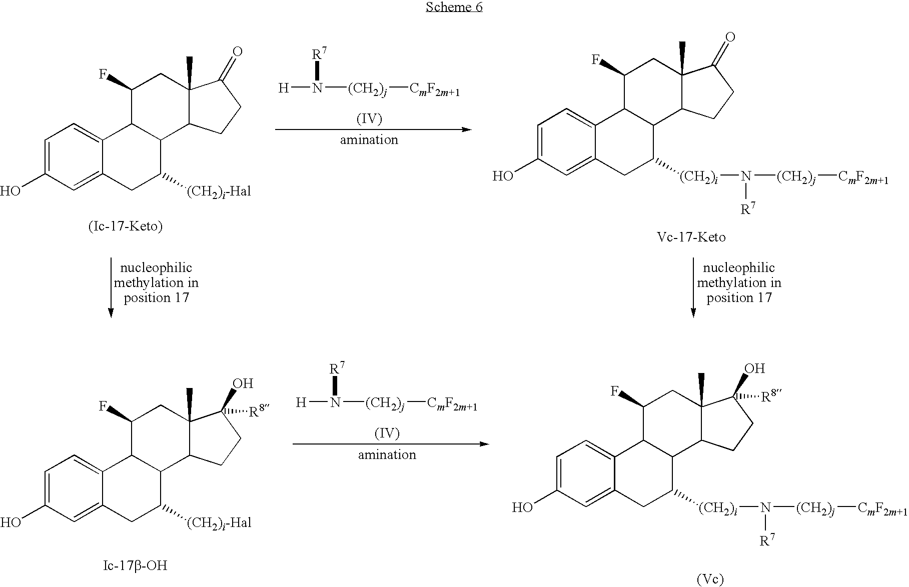

where

is an integer from 3 to 13,

Hal is a chlorine, bromine or iodine atom,

R^{8″} is a methyl group,

R⁷ is a C₁-C₄-alkyl radical,

j is an integer from 1 to 10 and

m is an integer from 1 to 5.

Accordingly, compounds of the formula (Ic-17-Keto) can first be reacted with α-alkyl(amine)-ω-perfluoro(alkyl)alkanes of the general formula (IV) to obtain compounds of the formula (Vc-Keto). Subsequently, the compounds of the formula (Vc-Keto) are converted in a nucleophilic alkylation reaction in position 17 to obtain compounds of the formula (Vc).

Alternatively and preferably, compounds of the formula (Ic-17-Keto) are first converted by a nucleophilic alkylation reaction in position 17 to compounds of the formula (Ic-17β-OH). Subsequently, the compounds of the formula (Ic-17β-OH) are aminated with α-alkyl(amine)-ω-perfluoro(alkyl)alkanes of the general formula (IV) to obtain compounds of the formula (Vc).

The methyl group in the 17α position can be introduced in each case by customary alkylating reagents, for example Grignard reagents (methyl-MgHal) or methyllithium.

A possible route to the aromatization of the A ring is disclosed in WO 99/33855:

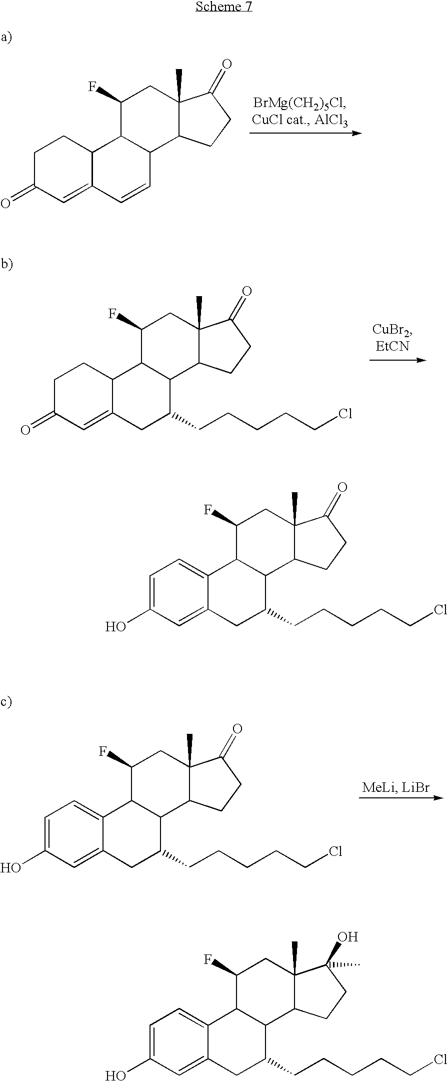

According to this prior art, the A ring is aromatized in partial step b) by a CuBr₂-mediated oxidation.

A disadvantage of the performance of partial step b) according to WO 99/33855 is that, under the reaction conditions known to date, a subsequent reaction forms brominated by-products which reduce the yield of compounds of the formula (I) and are difficult to remove from the product.

One example is the following side/subsequent reaction:

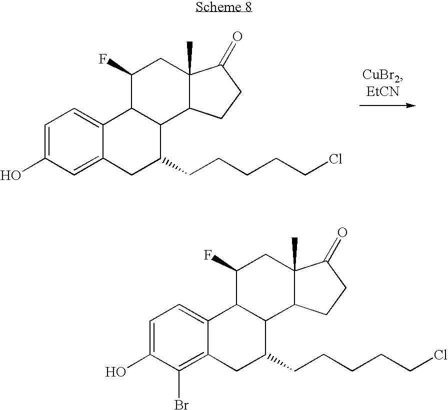

The bromination product is an undesired impurity, and has to be removed later in the synthesis—either by complicated chromatography or by repeated crystallization of an intermediate or of the active ingredient.

The literature (WO 2007/049672 and Steroids 1994, vol. 59, p. 621ff.) discloses that comparable oxidations of steroidal systems of the general type (11) to steroidal systems of the general type (I) can be carried out using different amounts of CuBr₂.

The subsequent or side reaction is also described in WO 02/32922. To minimize the bromination, a process is proposed there with addition of acetic anhydride. However, the process according to WO 02/32922 has the following disadvantages:
it is necessary to work with more than 2 equivalents of CuBr₂ (2 equivalents=stoichiometric), i.e. an air oxidation is not utilized, as a result of which the process becomes costly,
the desired “free phenol” is not formed, but rather the 3-acetate. This 3-acetate subsequently has to be hydrolysed to the desired product in a further reaction step,
the removal of the large amounts of Cu salts necessitates a further process step.

Proceeding from this prior art, it is an object of the present invention to provide a process for preparing compounds of the general formula (I) which suppresses the formation of brominated by-products or subsequent products, in order in particular to increase the yield, in order to avoid additional purification steps, and in order to reduce the amounts of copper-containing wastewater obtained.

In particular, brominated by-products of the formula (I-n) should be avoided

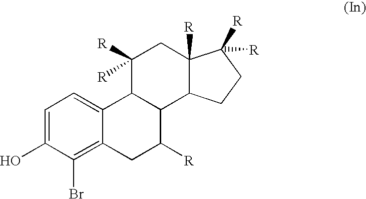

where each R is independently any chemically stable radical.

For instance, a minimum amount of by-products of the formula (Ia-n) should form in the preparation of compounds of the formula (Ia), a minimum amount of by-products of the formula (Ib-n) in the case of compounds of the formula (Ib), and a minimum amount of by-products of the formula (Ic-n) in the case of compounds of the formula (Ic).

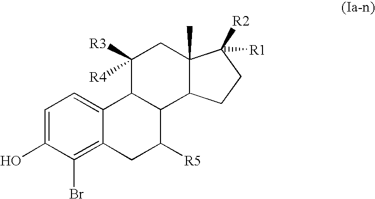

where R¹, R², R³, R⁴ and R⁵ are each as defined in formula (Ia)

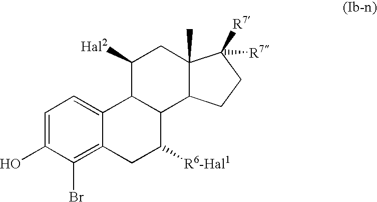

where R^{7′}, R^{7″}, R⁶-Hal¹ and Hal² are each as defined in formula (Ib)

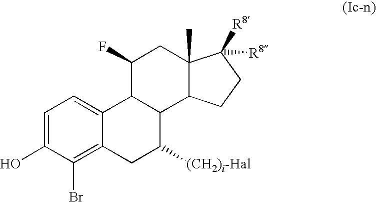

where i, R^{8′}, R^{8″} and Hal are each as defined in formula (Ic)

Furthermore, the CuBr₂ should have to be used in no more than stoichiometric amounts.

The process should preferably be accelerated by the addition of acids such that virtually complete to complete conversions can be achieved even with substoichiometric amounts of CuBr₂.

The oxidation should likewise preferably be performed in combination with an air oxidation.

The object of the present invention is achieved by effecting the CuBr₂-mediated aromatization of the rings A in the presence of at least one electron-rich, unsaturated organic additive.

Suitable electron-rich, unsaturated organic additives are substances of the general formula (Z)

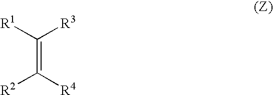

where
R¹, R², R³, R⁴ are each independently hydrogen, a C₁-C₆-alkyl, C₂-C₆-alkenyl, C₁-C₆-alkoxy or C₂-C₆-alkenoxy radical or a C₆-aryl ring or C₆-aryloxy ring, with the proviso that at least one radical of R¹, R², R³, R⁴ is not hydrogen,
   or
R¹ and R², together with the carbon atoms of the double bond, form a C₆-aromatic optionally mono- or polysubstituted identically or differently by hydroxyl, a C₁-C₆-alkoxy radical and/or C₁-C₆-alkyl radical, a C₃-C₇-cycloalkene, a monocyclic heteroaromatic or an unsaturated heterocycle optionally additionally substituted by R³ and R⁴, where R³ and R⁴ are each independently a C₁-C₆-alkyl, C₂-C₆-alkenyl, C₁-C₆-alkoxy or C₂-C₆-alkenoxy radical, or a C₆-aryl ring or C₆-aryloxy ring.

Preferably, R¹ and R² form C₆-aromatics polysubstituted by C₁-C₆-alkoxy radicals. This preferred subgroup of additives includes, as particularly preferred additives, 1,3,5-trimethoxybenzene and 1,3-dimethoxybenzene.

Preferably, R¹ and R² also form unsaturated cycloalkenes. This preferred subgroup of additives includes, as a particularly preferred additive, cyclohexene.

Preferably, R¹ and R² also form oxygen-containing unsaturated heterocycles. This preferred subgroup of additives includes, as a particularly preferred additive, dihydrofuran.

0.1 to 3 equivalents of the electron-rich unsaturated additive are used in relation to the steroid used, preferably 0.5 to 2.0 equivalents and more preferably 1 equivalent.

In general, the steroid is reacted together with a suitable amount of CuBr₂ and LiBr. This is done in a polar aprotic solvent, preferably acetonitrile or propionitrile. 1 equivalent of the steroid is reacted with 0.1 to 2.0 equivalents of CuBr₂ and 0.1 to 5.0 equivalents of LiBr. Preference is given to using 0.5 to 1.3 equivalents of CuBr₂ and 0.5 to 3.0 equivalents of LiBr. Particular preference is given to using 0.5 equivalent of CuBr₂ and 1.0 equivalent of LiBr.

In addition, a suitable amount of an acid of the general formula H⁺R⁻ is added—where R⁻ may be Hal⁻, —SO₂-alkyl, —SO₂-aryl.

Preference is given to methanesulphonic acid.

Also suitable are additives which release acids, for example trimethylsilyl bromide or trimethylsilyl chloride, from which HBr and HCl respectively are released.

The acid is added in an amount of 0.1 to 2.0 equivalents, preferably 0.2-0.6 equivalent.

For the complete oxidation of the reactant, air—preferably a mixture of nitrogen and oxygen—is subsequently passed through the reaction mixture until conversion is complete.

The process can be conducted at different temperatures, preferably in the range between +10° C. and +50° C.

An electron-rich unsaturated organic additive prevents the bromination of the steroid.

In the reaction mixture of Example 1, for example by means of HPLC analysis, the formation of 4-bromo-1-3-dimethoxybenzene and the decrease in 1,3-dimethoxybenzene can be monitored. It thus becomes clear that, caused by the different reactivities of the two electron-rich aromatics (steroidal phenol vs 1,3-dimethoxybenzene) with respect to a bromination, the more reactive 1,3-dimethoxybenzene is brominated first (see scheme 9) and the undesired bromination of the steroidal aromatic system is suppressed. The desired steroidal product is stable in the reaction mixture.

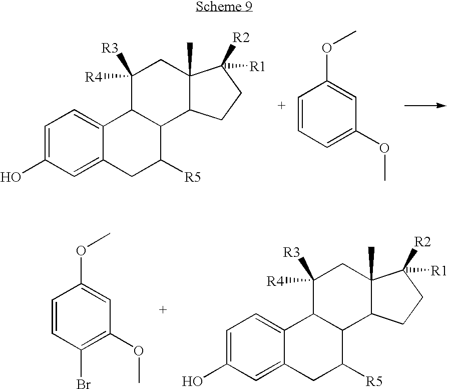

In the literature, there are no examples to date in which an aromatization of steroidal systems of the general type (II) is carried out using CuBr₂ and in the presence of electron-rich unsaturated additives.

In this case, the reaction can be accelerated by the addition of an acid and combined with an air oxidation.

### EXAMPLES

### Example 1

### Synthesis of 7-α-(5-chloropentyl)-11-β-fluoro-3-hydroxyestra-1,3,5(10)-trien-17-one (I-1) from 7-α-(5-chloropentyl)-11-β-fluoro-3-hydroxyestr-4-ene-3,17-dione (II-1)

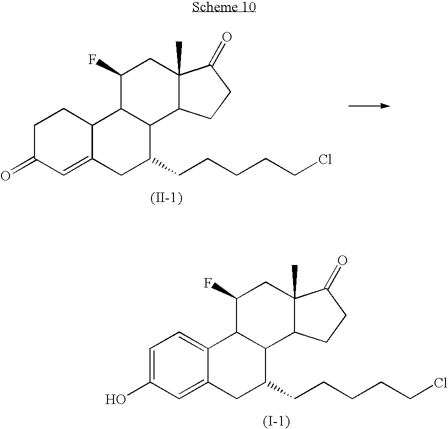

10.37 g (26.26 mmol) of 7-α-(5-chloropentyl)-11-β-fluoro-3-hydroxyestr-4-ene-3,17-dione are initially charged in 52 ml of propionitrile together with 3.42 ml (26.26 mmol) of 1,3-dimethoxybenzene. At RT, a solution of 2.93 g (13.13 mmol) of CuBr₂ and 2.28 g (26.26 mmol) of LiBr in 52 ml of propionitrile is added thereto. A mixture consisting of 70% (v/v) nitrogen and 30% (v/v) oxygen is passed through the reaction mixture. The reaction mixture is heated to 40° C. and admixed within 2 h with a solution of 0.85 ml (13.13 mmol) of methanesulphonic acid in 45 ml of propionitrile. The mixture is stirred until conversion is complete (approx. 4 h).

Subsequently, the reaction mixture is quenched by adding 40 ml of 20% aqueous K2HPO4 solution. During the continued stirring, the pH is monitored and, if appropriate, adjusted to pH=7. The suspension is filtered through a Celite-covered pressure filter which is washed with approx. 120 ml of toluene to free it of substance. After the water phase has been removed, the organic phase is extracted 2× with a solution consisting of 10 g of sodium edetate and 1 g of sodium hydroxide in 100 ml of water to remove the copper. The product solution is concentrated fully. 15.51 g of crude product are isolated. Composition of the crude product according to HPLC (Prontosil C18-ace-EPS; 1 ml/min water/ACN+0.1% HCOOH; 215 nm): 82.5% I-1, 0.0% I-2, 17.5% others.

Screening of Reaction Conditions and Scavengers on a Small Reaction Scale using II-1

In the development of the process described, a multitude of reaction conditions and possible scavengers were first studied on a small scale of only 100 mg of 7-α-(5-chloropentyl)-11-β-fluoro-3-hydroxyestr-4-ene-3,17-dione. On this reaction scale, even simple stirring in an open screwtop bottle in contact with ambient air is sufficient to bring about an oxidation, without any need for oxygen to be introduced specially. In screening experiments described below, this led to longer reaction times than in the finalized process, which, though, allows better observation of the course of the reaction. The suppression of secondary compounds through addition of 1,3-dimethoxybenzene can be shown clearly even by these screening experiments on a small scale. By way of example, results from two experiments are therefore reproduced. Under the conditions detailed above, by continuing removal of HPLC samples (100% method), a comparison of the reaction profiles with addition (reaction 1: Tab. 1, FIG. 1), and without addition (reaction 2: Tab. 2, FIG. 2), of 1,3-dimethoxybenzene is conducted.

For reaction 1 and reaction 2, 100 mg (0.25 mmol) of 7-α-(5-chloropentyl)-11-β-fluoro-3-hydroxyestr-4-ene-3,17-dione, 28 mg (0.13 mmol) of CuBr₂, 22 mg (0.25 mmol) of LiBr were initially charged in 1.5 ml of propionitrile, admixed with 16 μl (0.25 mmol) of methanesulphonic acid and stirred in an open vessel. Reaction 1 was additionally admixed with 99 μl (0.75 mmol) of 1,3-dimethoxybenzene. HPLC samples were taken at the given times. The analytical results are reproduced in the tables which follow:

TABLE 1
HPLC data for reaction 1

| Reaction time in | | | | | |
|---|---|---|---|---|---|
| hours | | I-1 | II-1 | I-1-n | Others |
| [h] | | [%] | [%] | [%] | [%] |
| 0 | h | 36.5 | 59.7 | 0.0 | 3.8 |
| 1 | h | 62.0 | 34.1 | 0.0 | 3.9 |
| 2 | h | 76.3 | 19.1 | 0.0 | 4.6 |
| 4 | h | 92.7 | 3.2 | 0.0 | 4.1 |
| 8 | h | 95.8 | 0.2 | 0.6 | 3.4 |
| 12 | h | 95.5 | 0.0 | 1.0 | 3.5 |
| 16 | h | 95.2 | 0.0 | 1.6 | 3.2 |
| 20 | h | 94.4 | 0.0 | 1.8 | 3.8 |
| 36 | h | 93.3 | 0.0 | 2.8 | 3.8 |

TABLE 2
HPLC data for reaction 2

| Reaction time in | | | | | |
|---|---|---|---|---|---|
| hours | | I-1 | II-1 | I-1-n | Others |
| [h] | | [%] | [%] | [%] | [%] |
| 0 | h | 32.9 | 64.0 | 0.0 | 3.2 |
| 1 | h | 58.3 | 40.0 | 0.0 | 1.7 |
| 2 | h | 73.4 | 23.4 | 0.0 | 3.2 |
| 4 | h | 90.0 | 5.7 | 0.2 | 4.1 |
| 8 | h | 91.5 | 0.0 | 3.4 | 5.1 |
| 12 | h | 82.0 | 0.0 | 14.1 | 3.9 |
| 16 | h | 67.4 | 1.2 | 29.2 | 2.2 |
| 36 | h | 44.2 | 0.0 | 52.9 | 2.9 |

### Example 2

### Synthesis of 8-α-estron (I-2) from 4-estrene-3,17-dione (II-2)

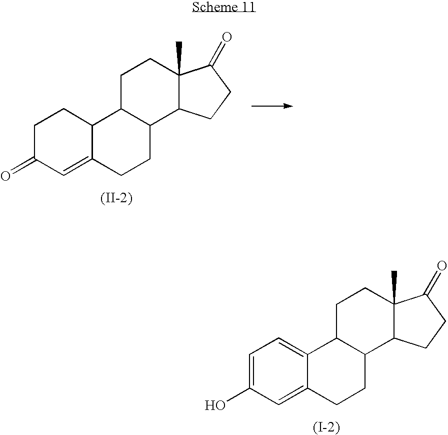

A solution of 69 mg (0.25 mmol) of 4-estrene-3,17-dione (II-2) in 1.5 ml of propionitrile is added at room temperature to 33 mg (0.14 mmol) of CuBr₂ and 28 mg (0.32 mmol) of LiBr in open 8 ml vials and shaken for 2 min. 0.5 ml of a solution of 4.9 mg (0.05 mmol) of methanesulphonic acid in propionitrile and 0.2 ml of a solution of 70 mg (0.51 mmol) of 1,3-dimethoxybenzene in propionitrile are then added. The reaction vessel is shaken while open—i.e. with contact of the reaction mixture with the ambient air—and samples for HPLC are taken. Composition of the reaction mixture after 8 h by HPLC (Phenomenex Synergi Polar-RP 4μ; 1 ml/min water/ACN+0.1% HCOOH; 220 nm): 91.7% I-2, 1.8% II-2, 6.5% others.

### Example 3

### Synthesis of 11-α-acetyloxy-3-hydroxyestra-1,3,5(10)-trien-17-one (I-3) from 11-α-acetyloxyestr-4-ene-3,17-dione (II-3)

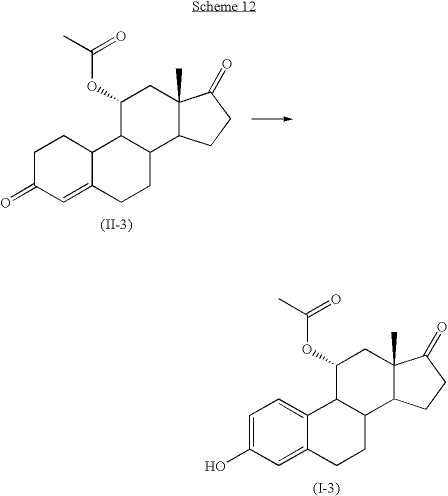

A solution of 84 mg (0.25 mmol) of 11-α-acetyloxyestr-4-ene-3,17-dione (II-3) in 1.5 ml of propionitrile and 1.5 ml of dichloromethane is added at room temperature to 33 mg (0.14 mmol) of CuBr₂ and 28 mg (0.32 mmol) of LiBr in open 8 ml vials and shaken for 2 min. 0.5 ml of a solution of 4.9 mg (0.05 mmol) of methanesulphonic acid in propionitrile and 0.2 ml of a solution of 70 mg (0.51 mmol) of 1,3-dimethoxybenzene in propionitrile are then added. The reaction vessel is shaken while open—i.e. with contact of the reaction mixture with the ambient air—and samples are taken for HPLC. Composition of the reaction mixture after 6 h by HPLC (Phenomenex Synergi Polar-RP 4μ; 1 ml/min water/ACN+0.1% HCOOH; 220 nm): 93.1% I-3, 0.0% II-3, 6.9% others.

### Example 4

### Synthesis of 11-β-fluoro-3-hydroxyestra-1,3,5(10)-trien-17-one (I-4) from 11-β-fluoroestr-4-ene-3,17-dione (II-4)

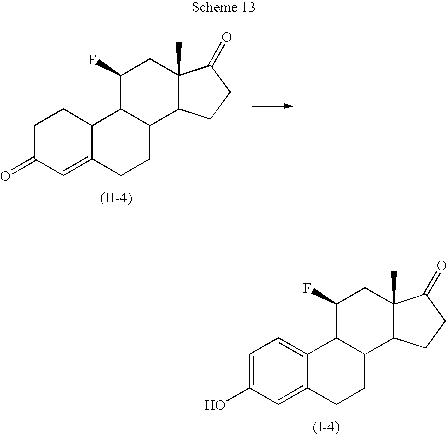

A solution of 74 mg (0.25 mmol) of 11-β-fluoroestr-4-ene-3,17-dione (II-4) in 1.5 ml of propionitrile is added at room temperature to 33 mg (0.14 mmol) of CuBr₂ and 28 mg (0.32 mmol) of LiBr in open 8 ml vials and shaken for 2 min. 0.5 ml of a solution of 4.9 mg (0.05 mmol) of methanesulphonic acid in propionitrile and 0.2 ml of a solution of 70 mg (0.51 mmol) of 1,3-dimethoxybenzene in propionitrile are then added. The reaction vessel is shaken while open—i.e. with contact of the reaction mixture with the ambient air—and samples for HPLC are taken. Composition of the reaction mixture at 18 h by HPLC (Phenomenex Synergi Polar-RP 4μ; 1 ml/min water/ACN+0.1% HCOOH; 220 nm): 78.0% I-4, 6.8% II-4, 15.2% others.

### Example 5

### Synthesis of 17-β-acetoxy-1,3,5(10)-estratrien-3-ol (II-5) from 17-β-acetoxy-4-estren-3-one (I-5)

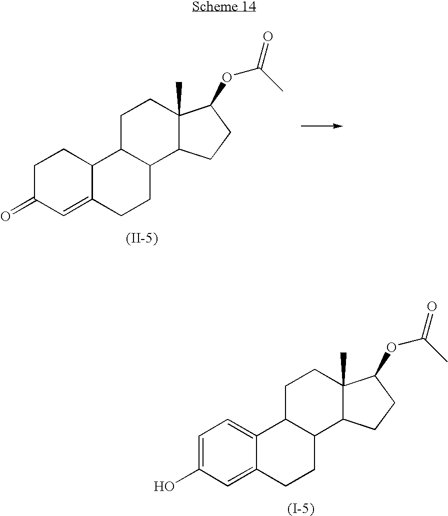

A solution of 80 mg (0.25 mmol) of 17-β-acetoxy-4-estren-3-one (II-5) in 1.5 ml of propionitrile is added at room temperature to 33 mg (0.14 mmol) of CuBr₂ and 28 mg (0.32 mmol) of LiBr in open 8 ml vials, and shaken for 2 min. 0.5 ml of a solution of 4.9 mg (0.05 mmol) of methanesulphonic acid in propionitrile and 0.2 ml of a solution of 70 mg (0.51 mmol) of 1,3-dimethoxybenzene in propionitrile are then added. The reaction vessel is shaken while open—i.e. with contact of the reaction mixture with the ambient air—and samples are taken for HPLC. Composition of the reaction mixture after 18 h by HPLC (Phenomenex Synergi Polar-RP 4μ; 1 ml/min water/ACN+0.1% HCOOH; 220 nm): 85.4% I-5, 2.1% II-5, 87.5% others.

### DESCRIPTION OF THE FIGURES

FIG. 1

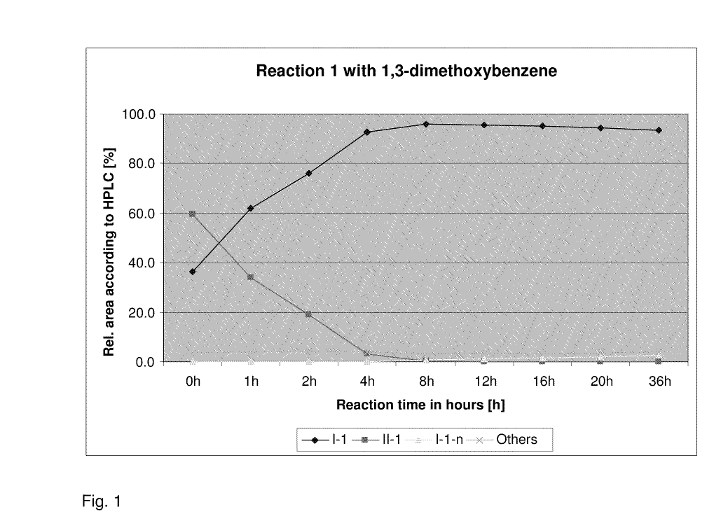

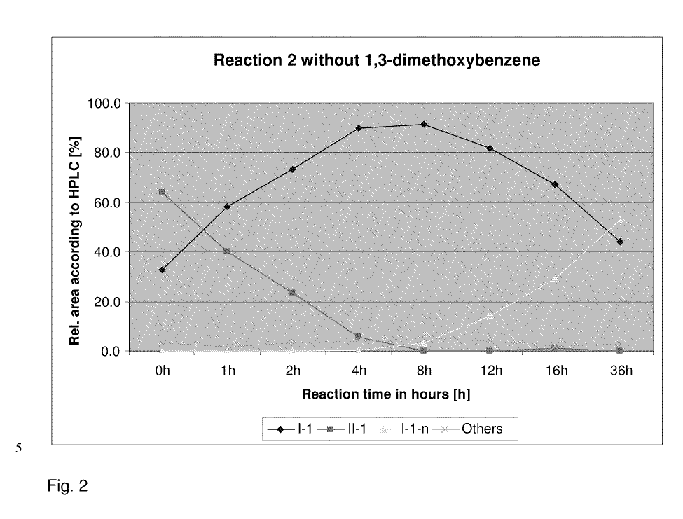

Graphic illustration of the HPLC data of reaction 1 from Tab. 1. Within the observation period of 36 h, no significant decomposition of the reaction product I-1 to the brominated by-product I-1-n is observed.

### =I-1;

### =II-2;

### =I-1-n,

[symbol] =others

FIG. 2

Graphic illustration of the HPLC data of reaction 2 from Tab. 2. Within the observation period of 36 h, significant decomposition of the reaction product I-1 to the brominated by-product I-1-n is observed.

### =I-1;

### =II-2;

### =I-1-n,

[symbol] =others

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 0707613.6, filed Dec. 13, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for CuBr₂-mediated aromatization of 19-norandrost-4-en-3-ones of the general formula (II) to estra-1,3,5(10)-trienes of the general formula I 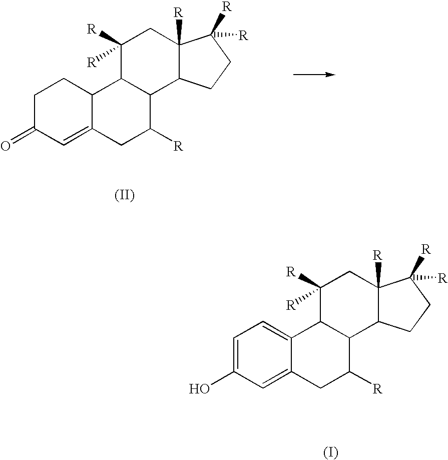
where, in formula (II) and formula (I), each R is independently any chemically stable radical, characterized in that the aromatization is effected in the presence of at least one electron-rich, unsaturated organic additive of the general formula (Z) 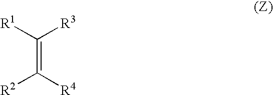
where
R¹, R², R³, R⁴ are each independently hydrogen, a C₁-C₆-alkyl, C₂-C₆-alkenyl, C₁-C₆-alkoxy or C₂-C₆-alkenoxy radical or a C₆-aryl ring or C₆-aryloxy ring, with the proviso that at least one radical of R¹, R², R³, R⁴ is not hydrogen,
or
R¹ and R², together with the carbon atoms of the double bond, form a C₆-aromatic optionally mono- or polysubstituted identically or differently by hydroxyl, a C₁-C₆-alkoxy radical and/or C₁-C₆-alkyl radical, a C₃-C₇-cycloalkene, a monocyclic heteroaromatic or an unsaturated heterocycle optionally additionally substituted by R³ and R⁴, where R³ and R⁴ are each independently a C₁-C₆-alkyl, C₂-C₆-alkenyl, C₁-C₆-alkoxy or C₂-C₆-alkenoxy radical, or a C₆-aryl ring or C₆-aryloxy ring,
and enantiomers and diastereomers thereof.

2. Process according to claim 1 for preparing estra-1,3,5(10)-trienes of the formula (Ia) from compounds of the formula (IIa) 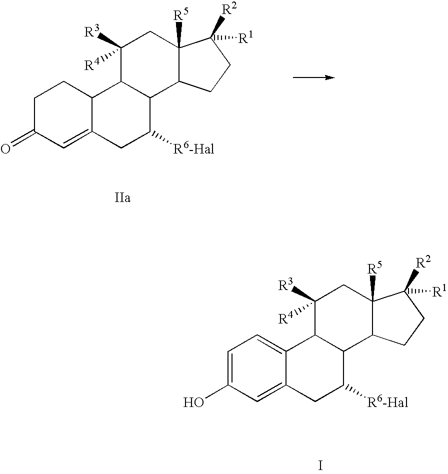
where
R¹ and R² together form a keto group,
or
R¹ is hydrogen, a C₁-C₆-alkyl, C₂-C₆-alkenyl or C₂-C₆-alkynyl radical, or is an at least partly fluorinated C₁-C₆-alkyl radical, and
R² is hydrogen, hydroxyl, C₁-C₆-alkoxy radical or a C₁-C₆-acyloxy radical;
R³ and R⁴ are each independently hydrogen, halogen and/or —OR^{B} where R^{B} is hydrogen or a protecting group,
R⁵ is a methyl or ethyl radical, and
R⁶-Hal is a C₃-C₁₃-alkyl, C₃-C₁₃-alkenyl or C₃-C₁₃-alkynyl radical, in each case at least monosubstituted by halogen,
and enantiomers and diastereomers thereof.

3. Process according to claim 1 for preparing compounds of the general formula (Ib) from compounds of the general formula (IIb) 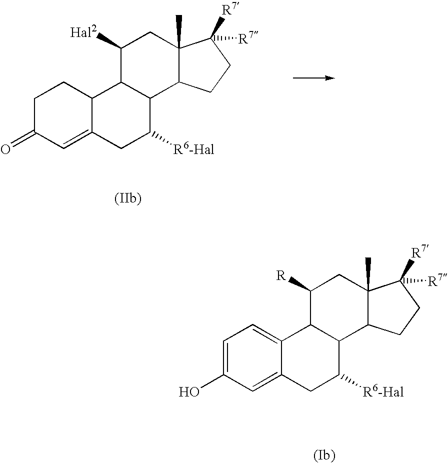
where
Hal² is F or Cl and is bonded to the estratriene base skeleton in the 11β position,
R^{7′} and R^{7″} together form a keto group,
or
R^{7′} is the —OR^{C} group where R^{C} is hydrogen or a protecting group,
R^{7″} is a C₁-C₄-alkyl radical or is an at least partly fluorinated C₁-C₄-alkyl radical,
where R^{7′} is bonded to the estratriene base skeleton in the 17β position and R^{7″} in the 17α position, and
R⁶-Hal¹ is bonded to the estratriene base skeleton in the 7α position and is a C₃-C₁₁-alkyl radical which is at least monosubstituted by Hal¹ where Hal¹ is a chlorine, bromine or iodine atom,
and enantiomers and diastereomers thereof.

4. Process according to claim 1 for preparing compounds of the general formula (Ic) from compounds of the general formula (IIc) 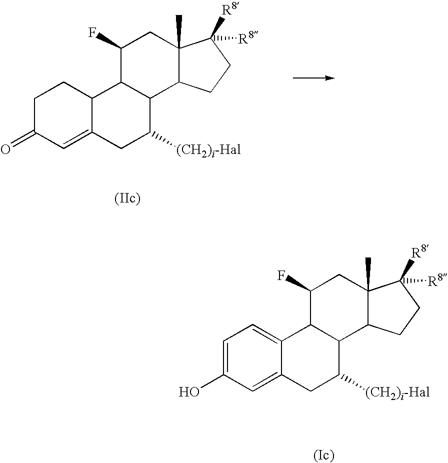
in which
R^{8′} and R^{8″} together form a keto group
or
R^{8′} is the —OR^{D} group where R^{D} is hydrogen or a protecting group,
R^{8″} is a methyl group,
i is an integer from 3 to 13 and
Hal is a chlorine, bromine or iodine atom,
and enantiomers and diastereomers thereof.

5. Process according to claim 1, wherein the additive used is 1,3,5-trimethoxybenzene and/or 1,3-dimethoxybenzene.

6. Process according to claim 1, wherein the additive used is cyclohexene.

7. Process according to claim 1, wherein the additive used is dihydrofuran.

8. Process for preparing compounds of the general formula (Vb), comprising the partial steps of
a) aromatizing a compound of the general formula (IIb-17-Keto) to a compound of the general formula (Ib-17-Keto) according to claim 3 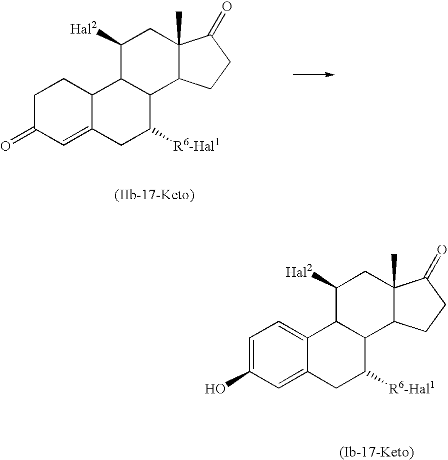
b) reacting the compounds of the general formula (Ib-17-Keto) with an amine of the formula H—W—X—Y-Z-E 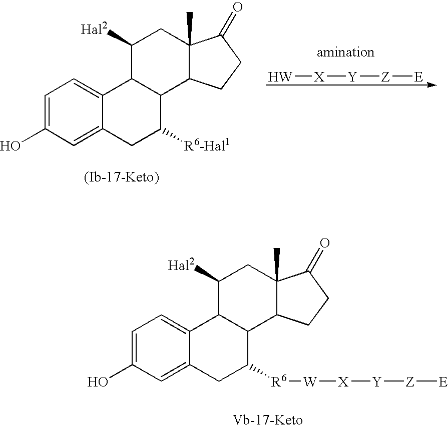
c) nucleophilically alkylating position 17 of the compounds of the formula (Vb-17-Keto) to obtain compounds of the formula (Vb) 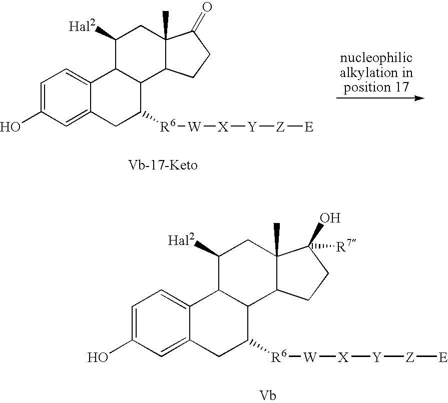
where Hal², R⁶—Hal¹ and R^{7″} are each as defined
and
W is —N(R⁷)— where R⁷ is hydrogen or a C₁-C₄-alkyl radical,
X is —(CH₂)_{q}— where q=0 or an integer of 1-12,
Y is a direct bond between X and Z or is a —SOₙ— group where n=0, 1 or 2,
Z is a straight- or branched-chain C₁-C₇-alkylene radical which is at least partly fluorinated,
E is —CF₃ or is pentafluorophenyl.

9. Process for preparing compounds of the general formula (Vb), comprising the partial steps of
a) aromatizing a compound of the general formula (IIb-17-Keto) to a compound of the general formula (Ib-17-Keto) according to claim 3 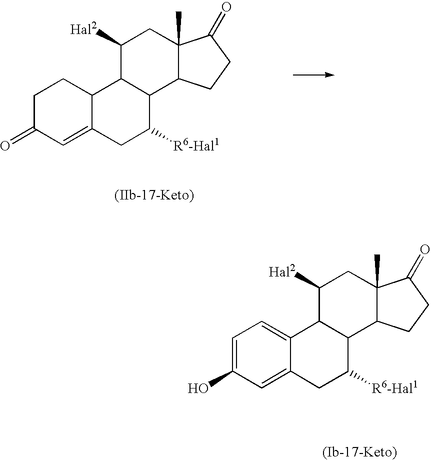
b) nucleophilically alkylating position 17 of the compounds of the formula (Ib-17-Keto) to obtain compounds of the formula (Ib-17β-OH) 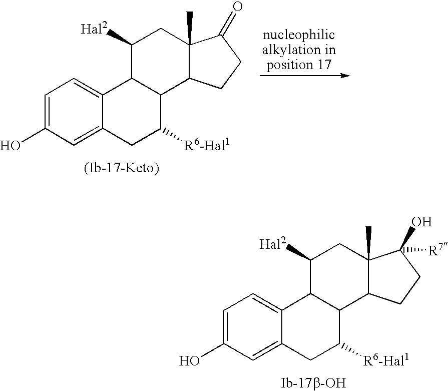
c) reacting the compounds of the general formula (Ib-17β-OH) with an amine of the formula H—W—X—Y-Z-E to obtain compounds of the formula (Vb) 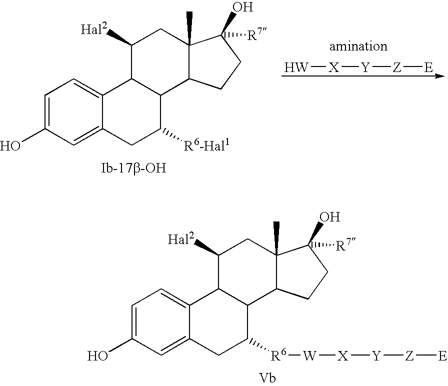
where Hal², R⁶—Hal¹ and R^{7″} are each as defined
and
W is —N(R⁷)— where R⁷ is hydrogen or a C₁-C₄-alkyl radical,
X is —(CH₂)_{q}— where q=0 or an integer of 1-12,
Y is a direct bond between X and Z or is a —SOₙ— group where n=0, 1 or 2,
Z is a straight- or branched-chain C₁-C₇-alkylene radical which is at least partly fluorinated,
E is —CF₃ or is pentafluorophenyl.

10. Process for preparing compounds of the general formula (Vc) comprising the partial steps of
a) aromatizing a compound of the general formula (IIc-17-Keto) to a compound of the general formula (Ic-17-Keto) according to claim 4 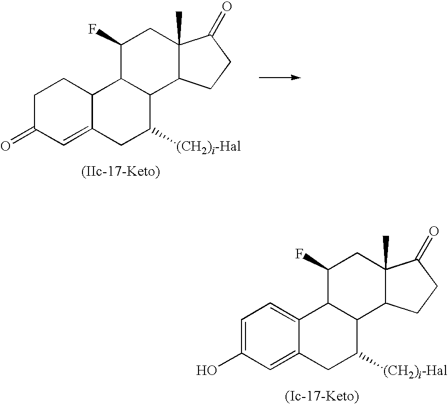
b) subsequently reacting the compounds of the general formula (Ic-17-Keto) with an α-alkyl(amine)-ω-perfluoro(alkyl)alkane of the general formula (IV) to give a compound of the general formula (Vc-Keto) 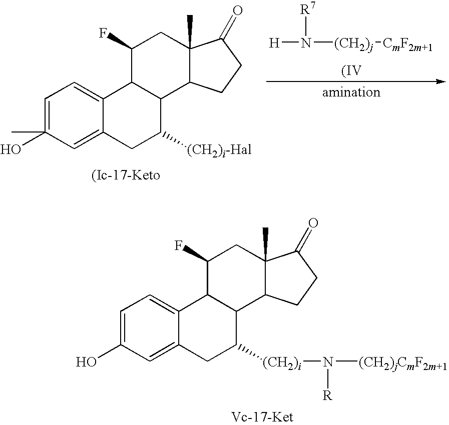
c) nucleophilically methylating position 17 of the compounds of the formula (Vc-17-Keto) to obtain compounds of the formula (Vc) 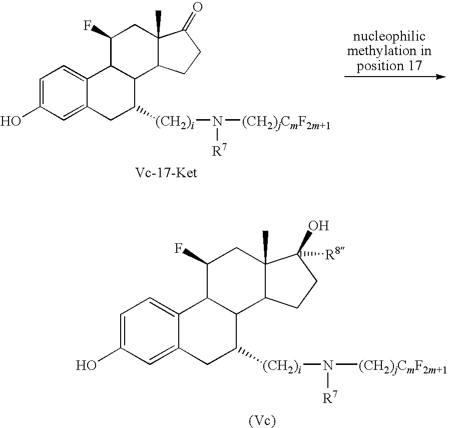
where
R^{8″} is a methyl group,
i is an integer from 3 to 13 and
Hal is a chlorine, bromine or iodine atom,
R⁷ is a C₁-C₄-alkyl radical,
j is an integer from 1 to 10 and
m is an integer from 1 to 5.

11. Process for preparing compounds of the general formula (Vc) comprising the partial steps of
a) aromatizing a compound of the general formula (IIc-17-Keto) to a compound of the general formula (Ic-17-Keto) according to claim 4 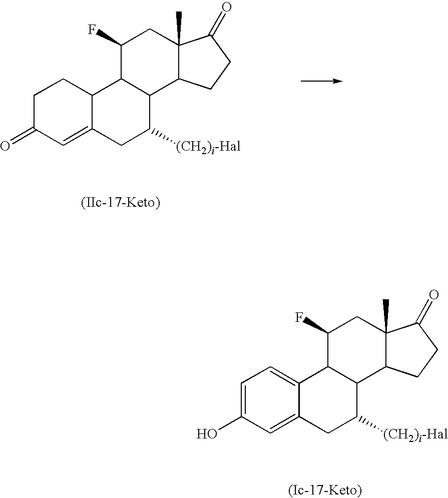
b) subsequently nucleophilically methylating position 17 of the compounds of the formula (Ic-17-Keto) to obtain compounds of the formula (Ic-17β-OH) 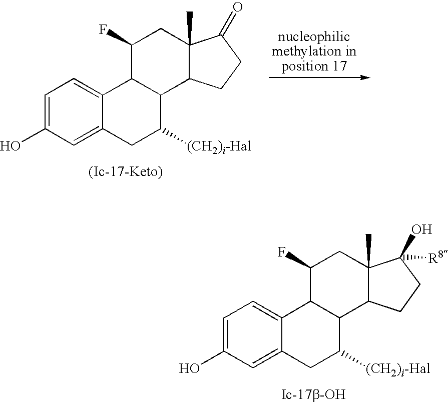
c) subsequently reacting the compounds of the general formula (Ic-17β-OH) with an α-alkyl(amine)-ω-perfluoro(alkyl)alkane of the general formula (IV) to give a compound of the general formula (Vc) 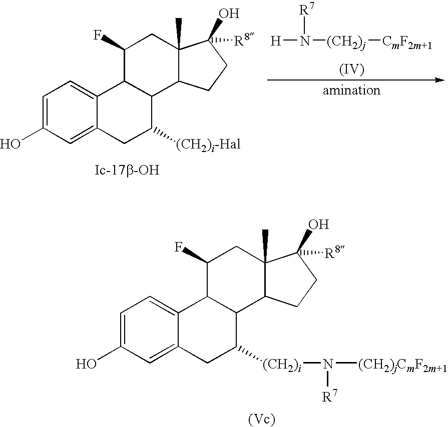
where
R^{8″} is a methyl group,
i is an integer from 3 to 13 and
Hal is a chlorine, bromine or iodine atom,
R⁷ is a C₁-C₄-alkyl radical,
j is an integer from 1 to 10 and
m is an integer from 1 to 5.
